(19) United States
(12) Patent Application Publication
(10) Pub. No.: US 2009/0022789 A1
(43) Pub. Date: Jan. 22, 2009

(54) ENHANCED FORMULATIONS OF LAMOTRIGINE

(75) Inventors: Argaw KIDANE, Montgomery Village, MD (US); Kevin Edwards, Lovettsville, VA (US); Padmanabh P. Bhatt, Rockville, MD (US)

Correspondence Address: FOLEY AND LARDNER LLP;SUITE 500, 3000 K STREET NW, WASHINGTON, DC, 20007, US

(73) Assignee: Supernus Pharmaceuticals, Inc., Rockville, MD (US)

(21) Appl. No.: 11/779,562

(22) Filed: Jul. 18, 2007

(51) Int. Cl. A61K 9/56; A61K 31/53; A61P 25/00; A61K 9/30

(52) U.S. Cl. 424/457; 424/475; 514/242

(57) ABSTRACT

A once-a-day, extended-release formulation of lamotrigine, exhibiting a significantly similar release rate throughout the GI tract irrespective of the pH of the environment, is provided. The formulation comprises lamotrigine, an organic acid, a release enhancing polymer and a release controlling polymer. The use of the formulation for the treatment of the neurological disorders is also disclosed.

### BACKGROUND OF THE INVENTION

Lamotrigine, having the chemical name 3,5-diamino-6-(2,3-dichlorophenyl)-as-triazine, is an anti-epileptic drug of the phenyltriazine class. The drug is typically used in both monotherapy and in adjunctive treatment with other antiepileptic agents for partial seizures and primary and secondary generalized tonic-clonic seizures in both adult and pediatric patients (i.e., children≧2 years of age). Lamotrigine is also indicated for seizures associated with the Lennox-Gastaut syndrome and as maintenance treatment of Bipolar I Disorder to delay the time to occurrence of mood episodes (e.g. depression, mania, hypomania, mixed episodes) in patients treated for acute mood episodes with standard therapy.

Lamotrigine is currently available as an immediate-release (“IR”) tablet as well as a chewable, dispersible tablet in various strengths from GlaxoSmithKline under the brand name Lamictal™. In this form, lamotrigine is rapidly absorbed after oral administration with negligible first-pass metabolism (absolute bioavailability is 98%). Lamotrigine is metabolized predominantly by glucuronic acid conjugation, the major metabolite of lamotrigine being an inactive 2-N-glucuronide conjugate. IR formulations of lamotrigine, however, cause side effects associated with a rapid rise in the blood concentration of the drug and/or level of exposure.

“Modified”-release doses of lamotrigine (i.e., doses other than IR, such as extended-release) could alleviate at least some of the side effects associated with immediate-release dosages. Because lamotrigine exhibits a decrease in solubility with increasing pH, the development of “modified”-release dosage forms of the drug has been problematic. When the release rate of a drug varies significantly with pH, the performance of a dosage form of the drug in vivo is influenced by the conditions of the gastrointestinal tract and the residence time of the dosage form in the different segments of the gastro-intestinal tract. In order to improve the release of drugs such as lamotrigine, the conventional wisdom has been to use acidifying agents, such as citric acid, in the formulation. However, this approach fails to enhance the release of lamotrigine in buffer media with pH greater than or equal to 6.8.

Hence, there is a need for a formulation of lamotrigine that exhibits a significantly similar release rate throughout the GI tract, irrespective of the pH of the environment. Advantageously, the formulation is an extended release formulation of lamotrigine; in particular, an extended release formulation of lamotrigine suitable for once-a-day administration.

### SUMMARY OF THE INVENTION

The current invention addresses some of the aforementioned needs, in part, by providing a pharmaceutical formulation comprising lamotrigine admixed with a release equalizing composition. In one embodiment, such a composition comprises a combination of a release-enhancing polymer and an organic acid.

In another embodiment of the invention, the lamotrigine formulation is an extended-release formulation. Extended release of lamotrigine may be achieved by incorporating a release-controlling polymer into the formulation. The resulting formulation exhibits a significantly similar release rate throughout the GI tract irrespective of the pH of the environment. The extended-release formulations of lamotrigine may be suitable for once-a-day administration, and take a variety of dosage forms, including tablets, pills, capsules, caplets, troches, sachets, cachets, pouches, and sprinkles.

Additionally, a method of treatment of a neurological disorder in a mammalian subject is disclosed that comprises administering to said subject any formulation of lamotrigine covered by the current invention.

### BRIEF DESCRIPTION OF THE DRAWINGS

FIG. 1 shows the pH solubility profile for lamotrigine.

FIG. 2 shows the solubility of lamotrigine in the presence of select excipients.

FIG. 3 shows the dissolution profiles of control lamotrigine IR pellets (Formulation B) in acid (pH 1.1) and phosphate buffer (pH 6.8) dissolution media.

FIG. 4 shows the dissolution profiles of lamotrigine IR pellets (Formulation A) containing citric acid but not Eudragit L100-55.

FIG. 5 shows the dissolution profiles of Lamotrigine extended release tablets (Formulation C) according to one embodiment of the invention (containing enteric polymer and organic acid) in acid (pH 1.1), phosphate buffer (pH 6.8) dissolution media, and medium changeover where dissolution was carried out in an acid medium (pH 1.1) for the first 2 hours followed by changeover to phosphate buffer (pH 6.8) from 2-20 hours.

FIG. 6 shows the release profiles in phosphate buffer medium of three exemplary Lamotrigine extended release (CR-F, CR-M, and CR-S) prototypes.

FIG. 7 shows the human PK profiles of three exemplary prototypes (Lamotrigine CR-F, CR-M, and CR-S) whose dissolution profiles are shown in FIG. 6.

FIG. 8 shows in silico steady state PK profiles for the three exemplary prototypes (Lamotrigine CR-F, CR-M, and CR-S).

### DETAILED DESCRIPTION OF THE INVENTION

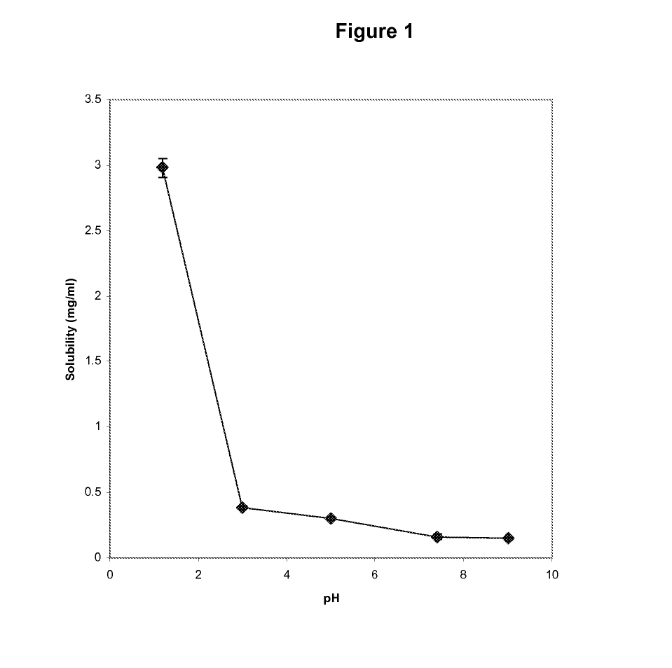

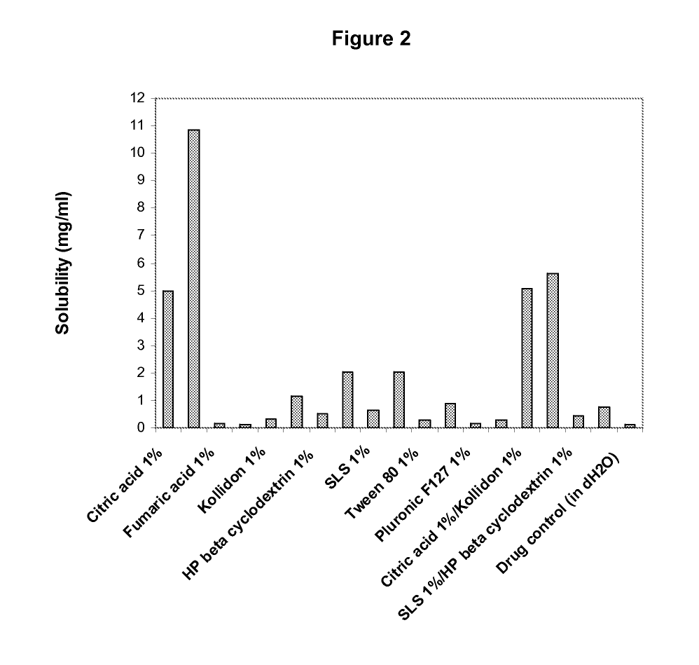

The solubility of lamotrgine is pH-dependent. FIG. 1 shows that lamotrigine is approximately 20 times more soluble at pH 1.2 than at pH 7.4 and FIG. 2 shows the effect of excipients on the drug's solubility. Taken together, these two figures suggest that the release rate of lamotrigine from a pharmaceutical formulation will be determined by or dependent upon pH. Hence, the “performance” (i.e., release rate) of a lamotrigine formulation in vivo will likely vary with the location of the formulation along segments of the GI tract—which have varying pH—and the residence time of the formulation in those segments. The variability of the release and performance is minimized by the formulations of the present invention.

As used herein, unless otherwise noted, “rate of release” or “release rate” of a drug refers to the quantity of drug released from a dosage form per unit time, e.g., milligrams of drug released per hour (mg/hr) or a percentage of a total drug dose released per hour. Drug release rates for dosage forms are typically measured as an in vitro rate of drug release, i.e., a quantity of drug released from the dosage form per unit time measured under appropriate conditions and in a suitable fluid.

The present inventors unexpectedly discovered that mixing lamotrigine with a “release-equalizing” composition, comprising a combination of a release-enhancing polymer and an organic acid, results in a lamotrigine formulation that exhibits (compared to an IR formulation of equivalent dose) enhanced solubility, enhanced dissolution and a significantly similar rate of release throughout the gastrointestinal (“GI”) tract, irrespective of the pH of the environment. A “release-equalizing” composition as defined herein is a composition that provides a significantly similar rate of release throughout the “GI” tract. For the purposes of this invention, the term “significantly similar release rate means that the similarity factor f₂ between the profile at the low pH and the profile at high pH is at least 50 (see “Guidance for Industry: Dissolution testing of Immediate Release Solid Oral Dosage Forms”, U.S. Department of Health and Human Services, Food and Drug Administration Center for Drug Evaluation and Research (CDER), August 1997, for the definition of f₂). The similarity factor (f₂) is a logarithmic reciprocal square root transformation of the sum of squared error and is a measurement of the similarity in the percent (%) dissolution between the two curves:

f₂=50 C log {[1+(1/n)3(R−T)] C 100}

Release-equalizing compositions of the present invention comprise a pharmaceutically acceptable organic acid and a release-enhancing polymer. Any pharmaceutically acceptable organic acid, as well as combinations of such acids, may be used in the practice of the current invention. Without any limitations thereon, suitable acids may be citric acid, fumaric acid, tartaric acid, adipic acid, succinic acid, and maleic acid, of which fumaric and citric acids are preferred. The acid may be incorporated into the release-equalizing formulation in the amount of from 1 to 20 wt %, preferably in the amount of from 5 to 20 wt %.

The release-enhancing polymers may be represented by enteric polymers soluble at pH≧4.5. Such polymers include, but are not limited to, cellulose acetate phthalate, cellulose acetate succinate, methylcellulose phthalate, ethylhydroxycellulose phthalate, polyvinylacetate phthalate, polyvinylbutyrate acetate, vinyl acetate-maleic anhydride copolymer, styrene-maleic monoester copolymer, methyl acrylate-methacrylic acid copolymer, and methacrylate-methacrylic acid-octyl acrylate copolymer. In a preferred embodiment of the invention, Eudragit L100-55 is used as a release-enhancing polymer. The release-enhancing polymers may be incorporated into the release-equalizing formulation in the amount of from 5 to 50 wt %; preferably in the amount of from 10 to 35 wt %.

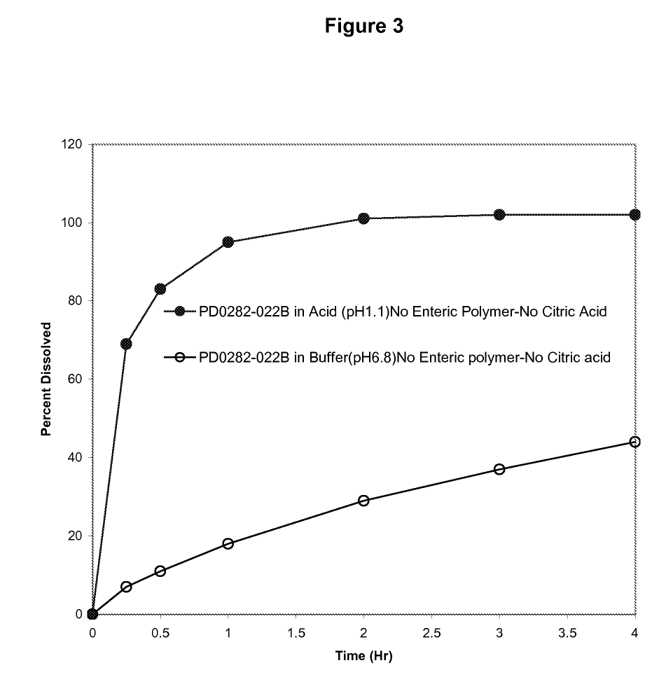

FIG. 3 shows dissolution profiles for formulations of lamotrigine without a release-equalizing composition in acid (pH 1.1) or phosphate buffer (pH 6.8) dissolution media. As expected, the profiles vary significantly with pH.

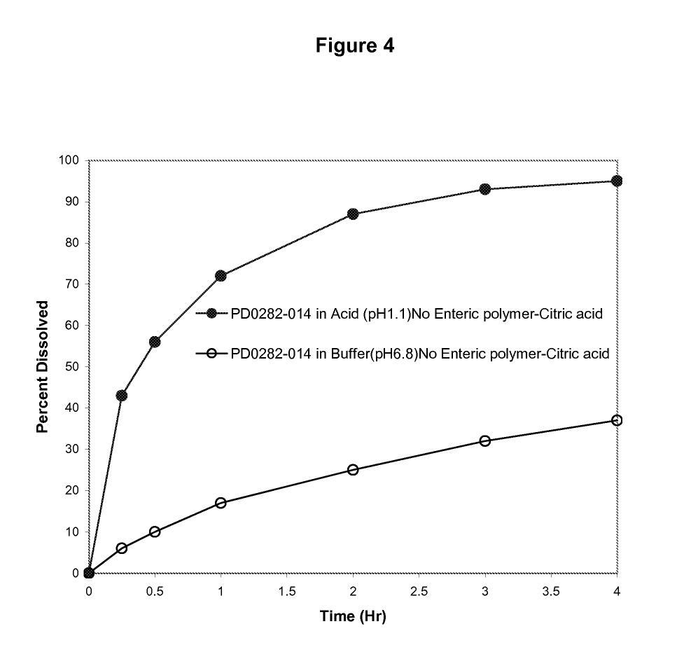

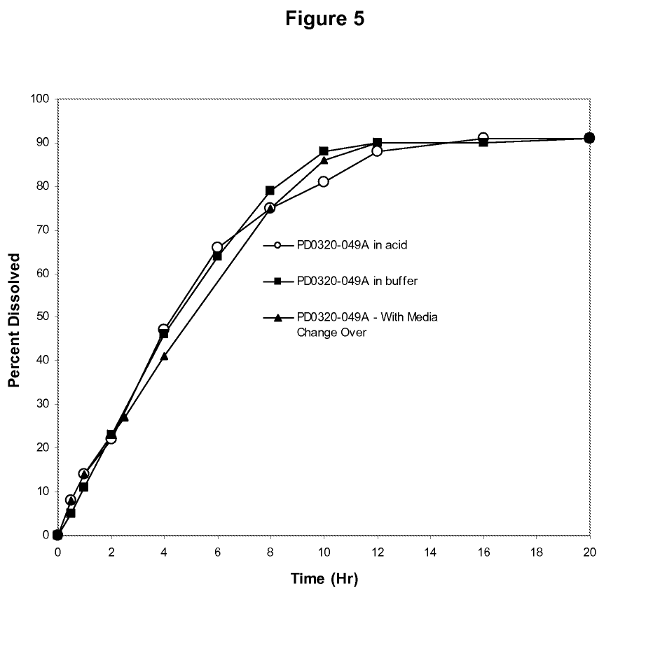

FIG. 4 shows dissolution profiles for lamotrigine in a release-equalizing composition that lacks a release-enhancing enteric polymer. There is a significant difference between the dissolution profile in acid (pH 1.1) versus phosphate buffer (pH 6.8) media. FIG. 5, by contrast, shows dissolution profiles for lamotrigine in release-equalizing formulations as taught by the present invention (i.e., containing both an organic acid and an enteric polymer). Note that the release of lamotrigine from this formulation is similar in either dissolution media, highlighting the pH-independence of the inventive formulations. Also shown in FIG. 5 is the dissolution profile for the same formulation with media changeover, where the dissolution is carried out in acid (pH 1.1) medium for the first 2 hours followed by media changeover to phosphate buffer (pH 6.8). The profile is similar to those obtained in acid and phosphate buffer media.

In a further embodiment of the invention, a “release-controlling” polymer may be additionally included into formulations of the current invention to provide “extended-release” formulations of lamotrigine that have a significantly similar release rate throughout the GI tract. An extended-release formulation (also referred to herein as “controlled release” or “CR”) is one that releases 80% of its drug in vitro (T₈₀) over a span of at least 2 hours.

Extended-release formulations of lamotrigine may have the release-controlling polymer(s) admixed with other ingredients into a release-equalizing formulation (e.g., organic acid and release-enhancing polymer), or may be supplied via a coating on a lamotrigine-containing matrix. Release-controlling polymers such as ethylcellulose, Eudragit RL, Eudragit RS, cellulose acetate, hydroxypropylmethylcellulose (HPMC), hydroxypropylcellulose (HPC), hydroxyethylcellulose (HEC), methylcellulose (MC), and PVA-PEG copolymer can be used to form an extended-release coating.

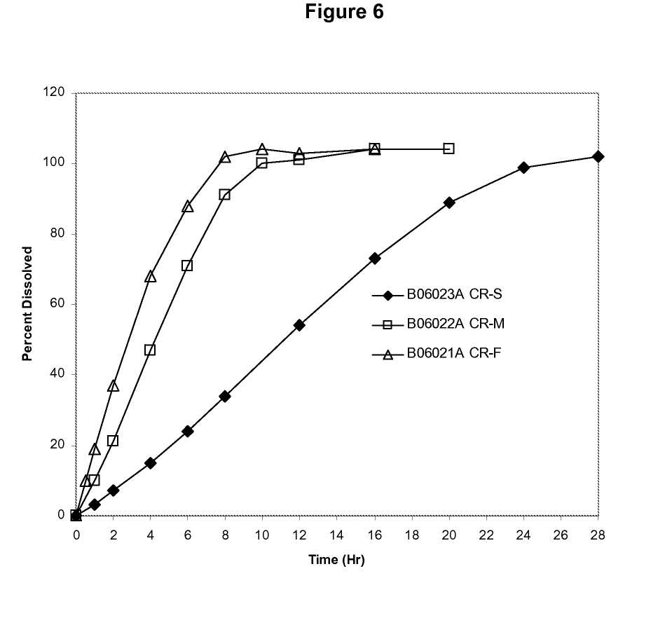

The present inventors also surprisingly discovered that when a release-controlling polymer is incorporated into a release-equalizing composition, the release-controlling polymer not only controls the rate of release, but also improves the effectiveness of the release-equalizing composition in a synergistic manner. The degree of synergism is determined by the nature and molecular weight of the release controlling polymer, as can be seen in Example 2 and FIG. 6, discussed below.

Polymers suitable for such synergistic action may be selected from a group consisting of hydroxypropylmethylcellulose (HPMC), hydroxypropylcellulose (HPC), hydroxyethylcellulose (HEC), methylcellulose (MC), powdered cellulose such as microcrystalline cellulose, cellulose acetate, sodium carboxymethylcellulose, calcium salt of carboxymethylcellulose, and ethylcellulose; alginates, gums such as guar and xanthan gums; cross-linked polyacrylic acid derivatives such as Carbomers (aka Carbopol™), that are available in various molecular weight grades from Noveon Inc. (Cincinnati, Ohio); carageenan; polyvinyl pyrrolidone and its derivatives such as crospovidone; polyethylene oxides; and polyvinyl alcohol.

The release-controlling polymer may be incorporated into the formulation in the amount of up to 50% by weight. In a particular embodiment of the invention, the release-controlling polymer is polyethylene oxide, preferably of the Polyox™class. Polyox™ are water soluble linear polyethylene oxide polymers available from Dow Chemical.

As may be seen from Examples 2 and 3 and FIG. 6, the release profile of the inventive lamotrigine compositions can be selectively adjusted by utilizing polyethylene oxide of different molecular weights. This level of adjustability provided by the current invention allows the development of extended-release formulations of lamotrigine that provide a better fit to the goals and modalities of treatment.

Extended-release formulations have an advantage over IR formulations in that the level of side-effects such as dizziness, ataxia, somnolence, headache, diplopia, blurred vision, nausea, vomiting, and rash, associated with rapid rise in the blood concentration of the drug when given in an IR form, is diminished. Fewer side-effects are known to lead to better treatment and compliance on the part of the patient.

In yet another embodiment, extended-release formulations of lamotrigine according to the invention provide a pharmacokinetic profile such that a maximum steady-state plasma concentration (Cmax) is in the range of from C_{minIR} to 110% of C_{maxIR}, wherein C_{minIR} and C_{maxIR} are the minimum and the maximum plasma concentrations respectively produced by the same amount of lamotrigine administered BID. In another embodiment, extended-release formulations of lamotrigine according to the invention provide a pharmacokinetic profile such that a relative steady state AUC is in the range of from 80% to 125% of an AUC_{IR}, wherein AUC_{IR} is an area under the curve produced by the same amount of lamotrigine administered as an immediate-release formulation BID.

For the purposes of this invention, BID administration is defined as administration of the same amount of lamotrigine in two equal doses twelve hours apart. Further, AUC is defined here as the area under the plasma concentration-time curve the AUC is directly proportional to the total amount of unmetabolized drug that reaches the systemic circulation.

Extended-release formulations of lamotrigine may be designed for once-a-day administration, though other modes of administration, such as twice-a-day, are also possible. The formulations of the current invention may be incorporated in any solid oral dosage form including, but not limited to, a tablet, a capsule, a caplet, a troche, a sachet, a cachet, a pouch, and sprinkles. The amount of lamotrigine in the dosage form may vary from 5 mg to 500 mg.

The formulations of the current invention may be used for the treatment or prevention of all disorders or conditions of a mammalian subject for which lamotrigine treatment is indicated or desirable. Without putting any limitations thereon, such disorders include epilepsy, Lennox-Gastaut syndrome, and bipolar disorder.

The formulations of the current invention may be prepared by a variety of methods well known in the art and include a variety of standard pharmaceutical excipients such as fillers, binders, lubricants, and others.

### EXAMPLES

### Example I

### Control Lamotrigine Immediate-Release (“IR”) Pellets

Table 1 provides the composition of two control formulations (Formulation A and Formulation B) of lamotrigine IR pellets. Formulations A and B were prepared by mixing dry components in KG-5 high shear granulator (Key International, Englishtown, N.J.). Granules were then extruded using DG-L2 Dome granulator (LCI Corporation, Charlotte, N.C.). Extrudates were spheronized using QJ-400G spheronizer (LCI Corporation, Charlotte, N.C.). Pellets were dried in an oven at 40° C. overnight. Dissolution tests were carried out in acid (pH 1.1) and phosphate buffer (pH 6.8) media. Dissolution profiles are shown in FIGS. 3 and 4. Both formulations exhibited pH dependent release profiles.

TABLE 1
Composition of two lamotrigine IR pellets (% w/w).

| | PD0282-014 | PD0282-022B |
|---|---|---|
| Components | Formulation A | Formulation B |
| Lamotrigine | 50 | 50 |
| SMCC50 | 30 | 45 |
| Lactose | 5 | |
| Kollidon 25 | 5 | 5 |
| Sodium Lauryl Sulfate | 5 | — |
| Maltodextrin (Maltrin | 5 | — |
| M150) | | |
| Citric Acid | 5 | — |
| Total | 100 | 100 |

### Example II

### Lamotrigine Tablet Composition of the Invention

Table 2 provides the composition of Formulation C according to the invention. The formulation was granulated using a Key high shear granulator with water as a granulating liquid. The drug and excipients were added to the bowl, and dry blended for about 1 minute at a blade speed of ˜260 RPM. A total of 112 g of deionized water was added to achieve granulation. The lot was dried in a fluid bed dryer (GPCG-1) to a final product temperature of no more than 50° C. The moisture content of the granulation was determined using a moisture analyzer, and was found to be 1.53% by weight. The dried granules were screened through an 18 mesh sieve, blended with magnesium stearate and tableted on a rotary tablet press. The target tablet weight and hardness were 333 mg and 8-10 kp, respectively. Tablet dissolution was tested in acid (pH 1.1), phosphate buffer (pH 6.8), and media change-over where the dissolution medium was changed from acid to buffer at the two hour time point during the dissolution run. The dissolution profiles are shown in FIG. 5.

TABLE 2
Composition for Lot PD0320-049A (Matrix Tablets).
Formulation C

| Component | (% w/w) |
|---|---|
| Lamotrigine | 30 |
| Prosolv SMCC50 | 10 |
| Eudragit L100-55 | 25 |
| Fumaric Acid | 5 |
| Polyox WSR N80 | 25 |
| Kollidon 25 | 5 |
| Magnesium Stearate | 0.5 |
| Batch Size (g) | 500 |

### EXAMPLE III

### Lamotrigine Extended Release (CR-F, CR-M, and CR-S) Clinical Batches

Three prototypes of the invention were manufactured to provide fast, medium and slow extended release profiles (Lamotrigine CR-F, CR-M, and CR-S, respectively). The composition for these prototypes is given in Table 3. These prototypes were manufactured following the same processing steps as provided in Example II. The products were used in a human pharmacokinetic (PK) study.

TABLE 3
Composition of Lamotrigine CR-F, CR-M and CR-S Clinical Batches

| | B06021 | | |
|---|---|---|---|
| | (CR-F) | B06022 (CR-M) | B06023 (CR-S) |
| Formulation | % w/w per dosage unit | | |
| Lamotrigine | 30.0 | 30.0 | 30.0 |
| Prosolv SMCC50 | 9.5 | 9.5 | 9.5 |
| Polyethylene Oxide, NF | 25.0 | — | — |
| (Polyox WSR N80) | | | |
| Polyethylene Oxide, NF | — | 25.0 | — |
| (Polyox WSR 1105) | | | |
| Polyethylene Oxide, NF | — | — | 25.0 |
| (Polyox WSR 301) | | | |
| Methacrylic Acid | 25.0 | 25.0 | 25.0 |
| Copolymer, Type C, NF | | | |
| (Eudragit L100-55) | | | |
| Fumaric Acid, Fine | 5.0 | 5.0 | 5.0 |
| Granular | | | |
| Povidone, USP | 5.0 | 5.0 | 5.0 |
| (Kollidon 25) | | | |
| Magnesium Stearate, NF | 0.5 | 0.5 | 0.5 |
| (PH Vegetable) | | | |
| Batch Size | 100.0 | 100.0 | 100.0 |

FIG. 6 shows the dissolution profiles for these prototypes in phosphate buffer medium (pH 6.8).

### Example IV

### Human Pilot Pharmacokinetic Study and in silico Modeling

Human pilot pharmacokinetic studies were conducted to assess the pharmacokinetics of the once-a-day, 100-mg dose of the three exemplary lamotrigine extended-release (CR-F, CR-M, CR-S) tablets, and a twice-a-day 50-mg dose of immediate release Lamictal® tablets (2×25 mg tablets), for a total daily dose of 100 mg, of under fasting conditions.

TABLE 4
Drug formulations

| Formulation | Dose | |
|---|---|---|
| Lamotrigine CR-F Tablets, 100 mg | 1 × 100 | mg |
| Lamotrigine CR-M Tablets, 100 mg | 1 × 100 | mg |
| Lamotrigine CR-S Tablets, 100 mg | 1 × 100 | mg |
| Lamictal ® Tablets, 25 mg | 2 × 25 | mg (BID) |

Blood samples (1×3 mL) were collected at time 0, the time of dose administration (within 30-minute window prior to dose) and at (for Treatments A, B, and C) Time 0.25, 0.5, 1, 1.5, 2, 3, 4, 5, 6, 8, 10, 12, 16, 18, 24, 36, 48, 60, 72 and 96 hours post-dose for the three exemplary controlled release products. Blood collection times for the reference product were at Time 0, the time of dose administration (within 30-minute window prior to dose), and at Time 0.25, 0.5, 0.75, 1, 1.5, 2, 2.5, 3, 4, 5, 6, 8, 11.833 (up to 10 minutes prior to the second dose administration), 12.5, 13, 13.5, 14, 14.5, 15, 16, 17, 18, 20, 24, 36, 48, 60, 72 and 96 hours post-first-dose. Plasma samples were analyzed using an LC-MS instrument.

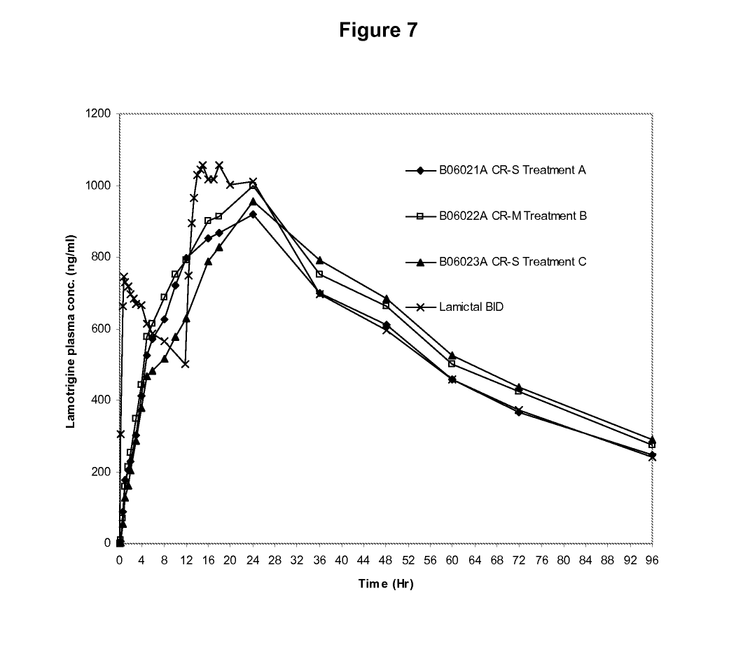

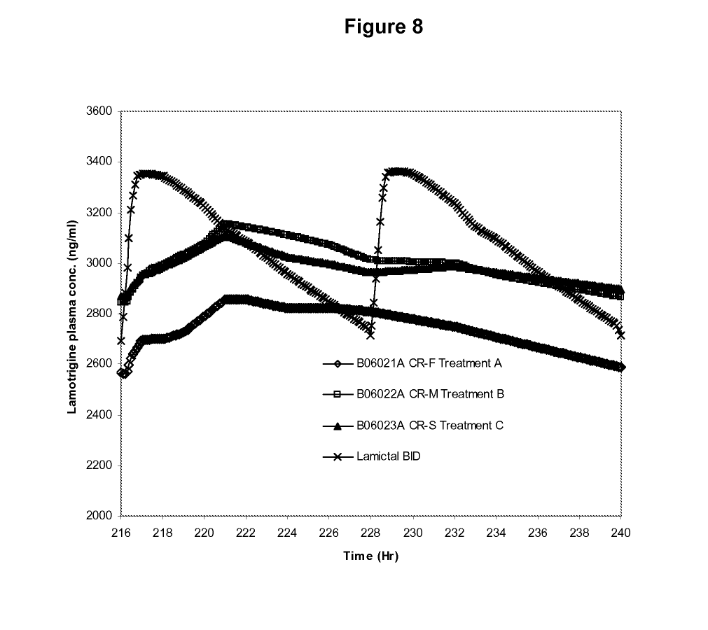

FIG. 7 shows the plasma lamotrigine concentration versus time profiles for the three extended release products and the reference product. Shown in FIG. 8 is the in silico steady state plasma lamotrigine concentration versus time profiles.

What is claimed is:

1. A pharmaceutical formulation of lamotrigine comprising lamotrigine or salts thereof admixed with a release-equalizing composition comprising a pharmaceutically acceptable organic acid and a release-enhancing polymer, said formulation exhibiting a significantly similar rate of release with a similarity factor of at least 50 throughout the GI tract.

2. The formulation of claim 1, wherein the release enhancing polymer is an enteric polymer.

3. The formulation of claim 2, wherein said enteric polymer is soluble at pH≧4.5.

4. The formulation of claim 3, wherein said enteric polymer is selected from the group consisting of cellulose acetate phthalate, cellulose acetate succinate, methylcellulose phthalate, ethylhydroxycellulose phthalate, polyvinylacetate phthalate, polyvinylbutyrate acetate, vinyl acetate-maleic anhydride copolymer, styrene-maleic monoester copolymer, methyl acrylate-methacrylic acid copolymer, and methacrylate-methacrylic acid-octyl acrylate copolymer.

5. The formulation of claim 2, wherein said enteric polymer is Eudragit L100-55.

6. The formulation of claim 1, wherein said organic acid is selected from the group consisting of citric acid, fumaric acid, tartaric acid, adipic acid, succinic acid, and maleic acid.

7. The formulation of claim 6, wherein said organic acid is citric acid or fumaric acid.

8. The formulation of claim 1 which is an extended-release formulation.

9. The formulation of claim 8, wherein said formulation provides for a maximum steady state plasma concentration (Cmax) in the range from C_{minIR} to 110% of C_{maxIR}, wherein C_{minIR} and C_{maxIR} are the minimum and the maximum plasma concentrations respectively produced by the same amount of lamotrigine administered as an immediate-release formulation BID.

10. The formulation of claim 8, wherein said formulation provides for a relative steady state AUC in the range of from 80% to 125% of an AUC_{IR}, wherein AUC_{IR} is an area under the curve produced by the same amount of lamotrigine administered as an immediate release formulation BID.

11. The formulation of claim 8 further comprising a coating of a release-controlling polymer selected from a group consisting of ethylcellulose, Eudragit RL, Eudragit RS, cellulose acetate, hydroxypropylmethylcellulose HPMC, hydroxyethylcellulose (HEC), methylcellulose (MC), and PVA-PEG copolymer.

12. The formulation of claim 8 wherein said release-equalizing composition further comprises a release-controlling polymer selected from a group consisting of hydroxypropylmethylcellulose (HPMC), hydroxypropylcellulose (HPC), hydroxyethylcellulose (HEC), methylcellulose (MC), powdered cellulose, cellulose acetate, sodium carboxymethylcellulose, calcium salt of carboxymethylcellulose and ethylcellulose; alginates, guar gum, xanthan gum; cross-linked polyacrylic acid derivatives; carageenan; polyvinyl pyrrolidone and its derivatives; polyethylene oxides; and polyvinyl alcohol.

13. The formulation of claim 12, wherein said release controlling-polymer is polyethylene oxide.

14. The formulation of claim 1 comprising from 5 to 500 mg of lamotrigine or salts thereof.

15. The formulation of claim 1, comprising from 5 to 20 wt % organic acid.

16. The formulation of claim 1, comprising from 5-50 wt % release-enhancing polymer.

17. The formulation of claim 12, comprising up to 50 wt % release-controlling polymer.

18. The formulation of claim 8 suitable for once-a-day administration.

19. An extended-release formulation of lamotrigine exhibiting a significantly similar release rate with a similarity factor of at least 50 throughout the GI tract irrespective of the pH of the environment, said formulation comprising lamotrigine or salts thereof, a release-equalizing composition, and a release-controlling polymer.

20. The formulation of claim 19, wherein the release-equalizing composition comprises an organic acid and a release-enhancing polymer and is admixed with lamotrigine to form a matrix.

21. The formulation of claim 20, wherein said release controlling polymer is admixed into the matrix and has a dual function of controlling a rate of release, and equalizing the release profile synergistically with the organic acid and the release enhancing polymer.

22. The formulation of claim 20, wherein said release controlling polymer is coated onto the matrix.

23. The formulation of claim 21, wherein said release controlling polymer is selected from a group consisting of hydroxypropylmethylcellulose (HPMC), hydroxypropylcellulose (HPC), hydroxyethylcellulose (HEC), methylcellulose (MC), powdered cellulose, cellulose acetate, sodium carboxymethylcellulose, calcium salt of carboxymethylcellulose and ethylcellulose; alginates, guar gum, xanthan gum; cross-linked polyacrylic acid derivatives; carageenan; polyvinyl pyrrolidone and its derivatives; polyethylene oxides; and polyvinyl alcohol.

24. The formulation of claim 23, wherein said release controlling polymer is polyethylene oxide.

25. The formulation of claim 22, wherein said release controlling polymer is selected from a group consisting of ethylcellulose, Eudragit RL, Eudragit RS, cellulose acetate, hydroxypropylmethylcellulose (HPMC), hydroxypropylcellulose (HPC), hydroxyethylcellulose (HEC), methylcellulose (MC), and PVA-PEG copolymer.

26. An oral dosage form comprising a formulation of lamotrigine exhibiting a significantly similar release rate with a similarity factor of at least 50 throughout the GI tract.

27. The dosage of claim 26, wherein said formulation comprises lamotrigine or salts thereof, a pharmaceutically acceptable organic acid and a release enhancing polymer admixed together and forming a matrix.

28. The dosage form of claim 27, wherein said formulation is an extended release formulation.

29. The dosage form of claim 28, wherein said formulation additionally comprises a release controlling polymer.

30. The dosage form of claim 29, wherein said release controlling polymer is admixed into the matrix and has a dual function of controlling a rate of release, and equalizing the release profile synergistically with the organic acid and the release enhancing polymer.

31. The dosage form of claim 29, wherein said release controlling polymer is coated onto the matrix.

32. The oral dosage of claim 28 suitable for once a day administration.

33. The dosage form of claim 26 selected from a group consisting of a tablet, a pill, a capsule, a caplet, a troche, a sachet, a cachet, a pouch, and sprinkles.

34. A method of treatment of a neurological disorder in a mammalian subject comprising administering to said subject a formulation of lamotrigine exhibiting a significantly similar release rate with a similarity factor of at least 50 throughout the GI tract irrespective of the pH of the environment.

35. The method of claim 34, wherein said neurological disorder is selected from a group consisting of epilepsy, Lennox-Gastaut syndrome, and a bipolar disorder.

36. The method of claim 34, wherein said formulation comprises lamotrigine or salts thereof, a pharmaceutically acceptable organic acid and a release enhancing polymer admixed together and forming a matrix.

37. The method of claim 34, wherein said formulation is an extended release formulation.

38. The method of claim 37, wherein said formulation additionally comprises a release controlling polymer.

39. The method of claim 38, wherein said release controlling polymer is admixed into the matrix and has a dual function of controlling a rate of release, and equalizing the release profile synergistically with the organic acid and the release enhancing polymer.

40. The method of claim 38, wherein said release controlling polymer is coated onto the matrix.

41. The method of claim 37, wherein said formulation is administered once a day.

42. An extended-release formulation of lamotrigine that exhibits a significantly similar release rate with a similarity factor of at least 50 throughout the GI tract irrespective of the pH of the environment, said formulation comprising a therapeutically effective amount of lamotrigine or salts thereof, fumaric acid, Eudragit L100-55, and polyethylene oxide, admixed together and forming a matrix.

43. The formulation of claim 42 for once a day administration.
